(19) United States
(12) Patent Application Publication
(10) Pub. No.: US 2008/0279806 A1
(43) Pub. Date: Nov. 13, 2008

(54) Dermal Filler Composition

(76) Inventor: Kang Seon Cho, Seoul (KR)

Correspondence Address: BIRCH STEWART KOLASCH & BIRCH, PO BOX 747, FALLS CHURCH, VA, 22040-0747, US

(21) Appl. No.: 12/095,885

(22) PCT Filed: Nov. 2, 2007
(86) PCT No.: PCT/KR07/05507

§ 371 Date: Jun. 2, 2008

(30) Foreign Application Priority Data
Dec. 13, 2006 (KR) 10-2006-0126815

(51) Int. Cl. A61F 2/10

(52) U.S. Cl. 424/78.02

(57) ABSTRACT

Disclosed herein is a dermal filler composition. The composition includes polymethylmethacrylate (PMMA), cross-linked dextran, hydroxypropyl methylcellulose (HPMC), and physiological saline or distilled water. The composition rapidly restores volume at application sites by injection, does not require pre-testing, such as allergic skin testing, because it does not cause severe allergic reactions, is cheap, and is not easily degraded or absorbed in the body, thus ensuring a long-lasting volume augmentation effect. Due to the characteristics described above, the composition facilitates volume correction requiring a large amount (20 cc or greater) of dermal filler such as in augmentation phalloplasty.

### TECHNICAL FIELD

The present invention relates to a dermal filler composition, and more particularly to a dermal filler composition including polymethylmethacrylate (PMMA), cross-linked dextran, hydroxypropyl methylcellulose (HPMC) and physiological saline or distilled water.

### BACKGROUND ART

Human soft tissue maintains its structure thanks to proteins such as collagen and elastin and the extracellular matrix, which includes glycosaminoglycans. Soft tissue defects resulting from trauma, congenital abnormalities or diseases have been restored or repaired by injecting biological tissue, grafting an autologous tissue, or injecting synthetic polymer into areas of soft tissue defects so as to augment the soft tissue.

Material similar to skin tissue, called dermal filler, is typically used in wrinkle removal or contour repair. Such material is injected into a specific defective area of the skin so as to augment the soft tissue. Dermal fillers are classified into two types according to their action mechanisms. One type functions to directly increase the tissue volume for soft tissue augmentation. This type of filler includes collagen and hyaluronic acid and so on as a major component. Another type functions to cause a foreign body reaction for a given period of time and thus induce endogenous collagen formation for a long period of time or permanently, as well as directly restoring the missing volume. Polymethylmethacrylate (PMMA) is known to have both action mechanisms.

An optimal dermal filler material should meet the several requirements that follow. It should not be of animal origin. It should rapidly exhibit its effects and maintain its effects for as long a time period as possible. It should be inexpensive and ensure natural and comfortable soft tissue augmentation.

Currently available dermal fillers are mainly composed of collagen or hyaluronic acid. Collagen-based dermal fillers include EVOLENCE 30 (ColBar LifeScience), which is a porcine-derived collagen, Zyderm and Zyplast (Inamed), which are composed of bovine collagen, and CosmoDerm and CosmoPlast (Inamed), which are composed of human collagen. Hyaluronic acid-based dermal fillers include Rofilan (Rofil/Philoderm), Perlane and Restylane (Medicis/Q-Med AB), Teosyal (Teoxane SA), and Surgiderm (Corneal Laboratoire). However, there are problems with dermal fillers composed of collagen or hyaluronic acid. They are expensive and effective only for a very short period of time, thereby limiting their clinical applications.

Filler products having a longer duration of effects include MATRIDEX (BioPolymer GmbH & Co.KG), which is composed of cross-linked dextran(DEAE sephadex) and hyaluronic acid. Dermal fillers consisting of cross-linked dextran and hyaluronic acid exhibit volume augmentation immediately after injection. In this case, the hyaluronic acid is degraded and absorbed over six to twelve months, and the resulting hollow space is filled with endogenous collagen, newly produced by the cross-linked dextran, which stimulates the body to produce new collagen. Hyaluronic acid is an intercellular cementing substance in the epidermis and the dermis, which is involved in intercellular adhesion and acts as a lubricating agent between cells. Artificially synthesized hyaluronic acid is used to make injectable dermal fillers. However, hyaluronic acid degrades within one year, and cross-linked dextran degrades within one or two years. Thus, hyaluronic acid-based dermal fillers have a short duration of volume augmentation effects and are very expensive due to the hyaluronic acid.

A filler product lasting longer than cross-linked dextran-based fillers is Artefill (Artes Medical), which is composed of polymethylmethacrylate (PMMA), and bovine collagen. Bovine collagen is purified, solubilized into a liquid state, mixed with PMMA, and injected below the dermis. This dermal filler exhibits volume augmentation immediately after injection. The injected collagen is degraded, absorbed into the body, and replaced by newly produced endogenous collagen in its place. The PMMA stimulates the new collagen growth. In this way, volume augmentation is maintained. However, since the used collagen is derived from the animal source bovine, the filler requires pre-testing for hypersensitivity, such as allergic skin testing, limiting its clinical applications. Also, the collagen material is very expensive, so that patients must bear a high economic burden. Further, collagen is degraded and absorbed into the body too quickly (completely absorbed within three to six weeks), thereby not ensuring volume maintenance through new collagen formation.

As described above, due to the problems in which they require pre-testing, such as allergic skin testing, before application, are expensive, or are rapidly degraded and absorbed by the body, and thus are short-lasting, conventional dermal fillers are difficult to apply to augmentation phalloplasty, which requires the injection of a large amount of a dermal filler. In fact, for penile enlargement, a dermal filler needs to be injected in an amount as large as 20 cc or greater. This incurs a high expense, which makes it difficult for anyone to receive dermal filler treatments and drives most patients to surgical operations such as implantation of autologous dermal fat grafts or stored dermis.

### DISCLOSURE

### Technical Problem

Accordingly, the present invention has been made keeping in mind the above problems encountered in the conventional dermal fillers, and an object of the present invention is to provide a novel dermal filler composition, which is injected below the dermis, thereby leaving no scar, rapidly restores volume at application sites and sustains the volume augmentation, and does not contain collagen, which can cause allergic reactions, thereby not requiring pre-testing, such as allergic skin testing.

Another object of the present invention is to provide a novel dermal filler composition, which, unlike conventional dermal fillers, which contain collagen or hyaluronic acid as a major component, is not easily degraded or absorbed in the body, thus ensuring stable longer-lasting volume augmentation, and is cheaper than conventional dermal fillers, particularly facilitating volume correction requiring a large amount (20 cc or greater) of a dermal filler, such as in augmentation phalloplasty.

### Technical Solution

In order to accomplish the above objects, the present invention provides a dermal filler composition including polymethylmethacrylate (PMMA), cross-linked dextran, hydroxypropyl methylcellulose (HPMC), and physiological saline or distilled water.

The present invention also provides a dermal filler composition including 20-70%(v/v) of a mixture of polymethylmethacrylate (PMMA) and cross-linked dextran in a volume ratio of 1:0.25-10, 30-80% (v/v) of physiological saline or distilled water, and 5-40 mg of hydrokypropyl methylcellulose (HPMC) per cc of a mixture of PMMA, cross-linked dextran and physiological saline or distilled water.

### Advantageous Effects

In accordance with the present invention, the dermal filler composition leaves no scar, and rapidly restores the volume at application sites, because it is injected below the dermis and does not require that a surgical incision be made in the skin. Also, the composition does not require pre-testing, such as allergic skin testing, because it does not contain collagen, which can cause allergic reactions, and is thus very beneficial in clinical applications. The composition is not easily degraded or absorbed into the body, unlike collagen or hyaluronic acid, thereby ensuring stable longer-lasting volume augmentation. Further, the composition is 10 to 30 times cheaper than conventional dermal fillers,

In particular, the composition is applicable in augmentation phalloplasty, requiring a large amount (20 cc or greater) of a dermal filler, thereby facilitating penile enlargement and replacing a surgical operation, such as the implantation of autologous dermal fat grafts or stored dermis.

### BEST MODE

Polymethylmethacrylate (PMMA), which is used as a major component in the dermal filler composition of the present invention, is a microsphere that is 30 to 120 μm in diameter. This is large enough not to be phagocyted by macrophages, and does not easily penetrate into surrounding areas, where fibrosis is induced by PMMA. When PMMA is injected into the skin, it directly increases volume, is not absorbed into the body, and remains for a long period of time or permanently at the application site, where it stimulates fibroblasts to produce collagen. The produced collagen wraps PMMA, and the encapsulated PMMA microspheres fill wrinkles or other recessed areas.

Examples of cross-linked dextran, which is another component of the dermal filler composition of the present invention, include DEAE Sephadex (Pharmacia Fine Chemicals). It is a microsphere that is 30 to 120 μm in diameter. When injected into the skin, cross-linked dextran directly increases volume, and in addition, it is not phagocyted by macrophages, and induces a foreign body reaction for a predetermined period of time to thus promote collagen formation, thereby providing long-lasting volume augmentation.

The PMMA and cross-linked dextran are preferably mixed in a volume ratio of 1:0.25-10. A higher amount of PMMA makes it difficult to inject the present dermal filler. A higher amount of cross-linked dextran hardens the injected site.

The mixture of PMMA and cross-linked dextran is preferably contained in an amount of 20-70% based on the total volume (100) of the dermal filler composition of the present invention. Physiological saline accounts for the remaining volume of the composition. Instead of physiological saline, sterile distilled water can be used. The physiological saline or sterile water may be adjusted to a desired pH value using an acid, such as lactic acid or phosphate, or a base.

Hydroxypropyl methylcellulose (HPMC) functions to facilitate dermal filler injection by maintaining the PMMA/cross-linked dextran mixture in a gel state. HPMC is properly added according to the conditions of application sites in an amount of 5-40 mg per 1 cc of a mixture of PMMA, cross-linked dextran and physiological saline or distilled water.

The HPMC may be substituted with other materials having the same function. Examples of such materials include sodium carboxymethylcellulose, chitosan, polyethylene glycol (PEG), polylactic glycolamide (PLGA), hyaluronic acid, and polyvinyl alcohol (PVA).

1. A dermal filler composition comprising polymethylmethacrylate (PMMA), cross-linked dextran, hydroxypropyl methylcellulose (HPMC) and physiological saline or distilled water.

2. A dermal filler composition comprising 20-70% (v/v) of a mixture of polymethylmethacrylate (PMMA) and cross-linked dextran in a volume ratio of 1:0.25-10; 30-80% (v/v) of physiological saline or distilled water; and 5-40 mg of hydroxypropyl methylcellulose (HPMC) per cc of a mixture of PMMA, cross-linked dextran and physiological saline or distilled water.
